(19) United States
(12) Patent Application Publication
(10) Pub. No.: US 2015/0150709 A1
(43) Pub. Date: Jun. 4, 2015

(54) ORTHOSIS

(71) Applicant: OTTO BOCK HEALTHCARE GMBH, Duderstadt (DE)

(72) Inventors: Boris Ljubimir, Korntal-Munchingen (DE); Alexander Schmitt, Niestetal (DE)

(73) Assignee: OTTO BOCK HEALTHCARE GMBH, Duderstadt (DE)

(21) Appl. No.: 14/406,822

(22) PCT Filed: Jun. 6, 2013
(86) PCT No.: PCT/EP2013/001665

§ 371 Date: Dec. 10, 2014

(30) Foreign Application Priority Data
Jun. 12, 2012 (DE) 10 2012 011 467.2

(51) Int. Cl. A61F 5/01

(52) U.S. Cl. CPC A61F 5/0127

(57) ABSTRACT

An orthosis including a supporting region that has fastening means for placing the orthosis on a limb, wherein the supporting region has opposing receiving sections, that the orthosis further includes pockets for receiving the receiving sections in the fastening means.

The invention an orthosis with a supporting region that has fastening means for fitting the orthosis on a limb.

Orthoses are used to stabilize limbs and can be arranged extending across a joint. Orthoses are used, among other things, to ensure or support a desired pattern of movement, to reinforce the supporting apparatus, to limit the range of movement of joints, or to apply a defined force to a limb. In order to secure an orthosis on a limb, fastening means are used which are generally designed as belts or straps. These belts or straps are secured on frame parts, for example by adhesive bonding or by riveting.

The object of the present invention is to make available an orthosis that is easy to produce and to clean and that ensures reliable securing of the orthosis on the limb.

According to the invention, this object is achieved by an orthosis having the features of the main claim. Advantageous embodiments and developments of the invention are disclosed in the dependent claims, in the description, and in the figures.

In the orthosis according to the invention, with a supporting region that has fastening means for fitting the orthosis on a limb, provision is made that the supporting region has receiving regions oriented opposite to each other, e.g. in the form of projections, and that pockets for receiving the receiving regions are arranged on the fastening means.

With the receiving regions or projections oriented opposite to each other, form-fit elements are formed on the dimensionally stable supporting region of the orthosis, which form-fit elements can be inserted into pockets that are arranged on the fastening means. The pockets permit, on the one hand, reliable securing of the fastening means on account of the oppositely directed orientation of the projections and, on the other hand, simple removal of the fastening means from the supporting region, since the receiving regions can be removed from the pockets and the fastening means detached. It is thus possible to wash the fastening means, for example, or to use alternative fastening means which, for example, are better adapted to the shape of the limb.

The fastening means is advantageously part of a padding or has a padding, which is arranged on the side of the orthosis facing toward the padding. This ensures, on the one hand, the securing of the orthosis on the limb and, on the other hand, the securing of the padding on the supporting region. The reversible arrangement of the padding on the supporting region has the advantage that the padding can also be exchanged and can be adapted to the wishes or circumstances of the particular patient.

The pockets are advantageously arranged medially and laterally, so as to permit easier fitting of the orthosis.

In a development of the invention, provision is made that the laterally arranged pockets and/or receiving regions are longer than the medially arranged pockets and/or receiving regions. This ensures that, when a strap is guided from the medial side to the lateral side, the forces acting in the circumferential direction have the effect that the laterally formed receiving regions cannot slide out of the pockets. The arrangement with deeper pockets and longer receiving regions on the lateral side has the effect that, even in the event of a twisting movement in the circumferential direction by the length of the medial receiving regions or medial pockets, and the lateral receiving regions still remain in the lateral pockets. This avoids the receiving regions being pulled out of the pockets.

The supporting region advantageously has a cup-like or gutter-like shape, such that substantially planar contact is obtained around the limb. By virtue of the cup-like or gutter-like shape, it is possible to simply place the orthosis onto the limb and then secure it on the limb via the fastening means. The cup-like or gutter-like shape can be provided with apertures in order to permit ventilation and, if appropriate, to reduce the weight.

The pockets can be connected to each other via a reversibly attachable connection element, said connection element preferably being guided over the supporting region so as to prevent slipping down of the pockets, and therefore of the fastening means, from the receiving regions. In a development of the invention, provision is made that the connection element is elastic and thus allows the pockets to be pretensioned toward each other. The pretensioning is achieved if the connection element is stretched before being secured on the pockets. The pockets are then pulled onto the receiving regions and held there with pretensioning.

The connection element can be part of the fastening means. For example, the connection element can form a pocket, for example a medially arranged pocket, such that the connection element can be guided externally over the supporting element. The receiving regions are arranged between the connection element and the fastening means and are pushed into the pocket formed by the connection element and the fastening means.

A fixing means for the connection element can be arranged on the outer side of at least one pocket, for example in the form of loop and hook areas. It is thereby possible for the connection element to be designed as a strap looping around the supporting region and the limb, which strap at the same time fixes the pockets on the supporting element. If the strap is elastic, the pretensioning of the pockets toward each other can be effected via the strap. Provision is also made that the connection element can be designed as a strap looping around the supporting region and the limb, without being secured on the outer side of a pocket via a fixing means.

In the fitted state, provision is made that the connection element forms a lateral to medial bridge across a gap between the receiving regions. The strap is thus guided from the medial pocket over the lateral pocket and back to the medial pocket or the medial projection, as a result of which a full loop is made around the limb. In the case of a gutter-like or cup-like shape, the gap between the receiving regions is the space into which the limb is inserted.

In its longitudinal extent, which generally corresponds to the longitudinal extent of the limb, the supporting region can be designed elastically about an axis parallel to the longitudinal extent, such that the supporting region permits adaptability in the medial direction and lateral direction. Deformations going beyond this are advantageously not permitted by the supporting region, such that the supporting region is safely supported and the orthosis is thus secured on the limbs.

In a development of the invention, provision is made that the orthosis also has a set-down region, which is connected to the supporting region and bears on the limb. The set-down region is advantageously arranged extending across a joint, such that, for example in the case of a foot orthosis, the set-down region is the region on which the foot is put down, while the supporting region is the region of the orthosis that bears on the lower leg.

The set-down region and the supporting region can be connected to each other in an articulated manner, or provision is alternatively made that a more or less rigid connection is present between both regions, wherein a movement of the set-down region relative to the supporting region is possible within the context of the material elasticity.

The receiving regions can be designed as projections. The receiving regions allow the pockets to be secured on the orthosis, by these regions being inserted into the pockets. One receiving region or projection or a plurality of receiving regions or projections can be provided medially and laterally, such that there is either one pocket or several pockets arranged medially and laterally. The receiving regions extend medially and laterally rearward from the supporting region, thereby allowing them to be received in the pockets. If the supporting region is gutter-like and has straight wall edges, the receiving regions are designed as projections that jut out and protrude from the edges. It is also possible that the respective receiving region is provided by a formation that adjoins the supporting region.

Illustrative embodiments of the invention are explained in more detail below with reference to the attached figures, in which:

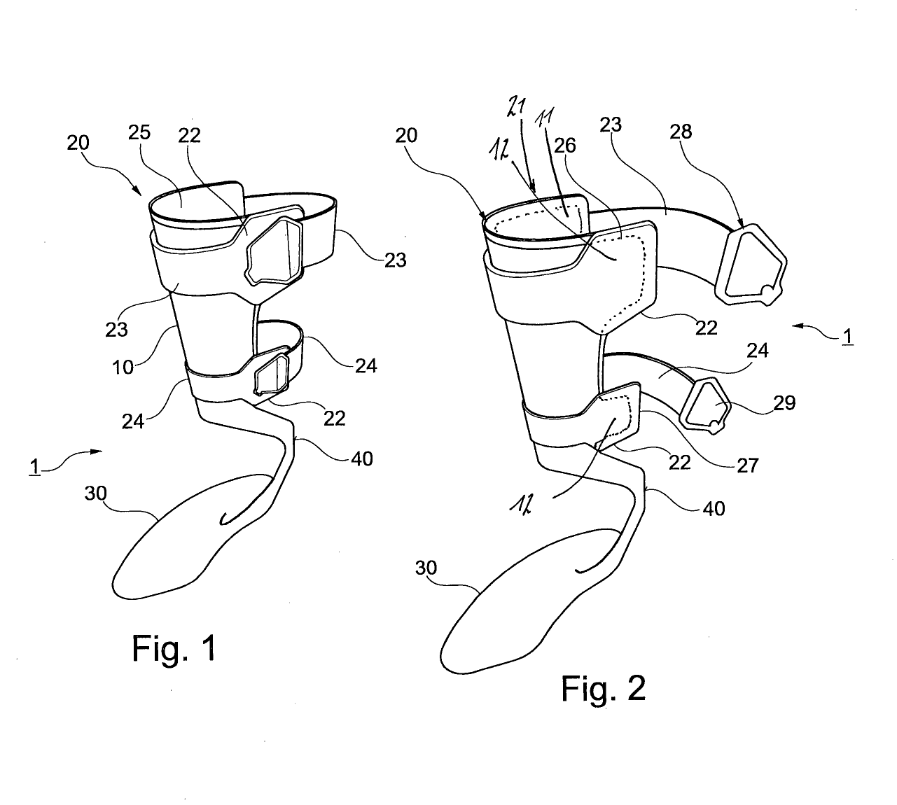

FIG. 1 shows an orthosis with closed connection elements; and

FIG. 2 shows an orthosis according to FIG. 1 with open connection elements;

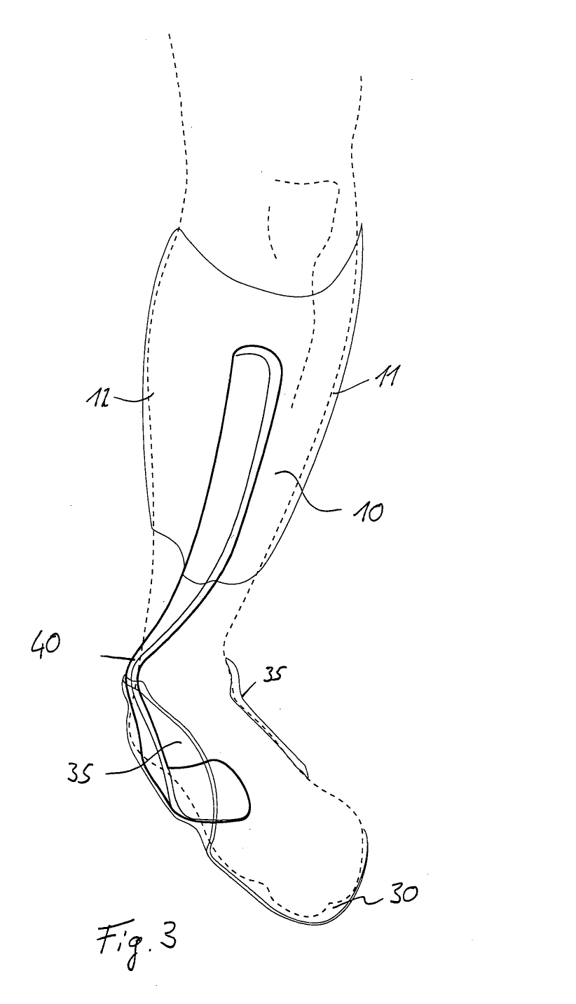

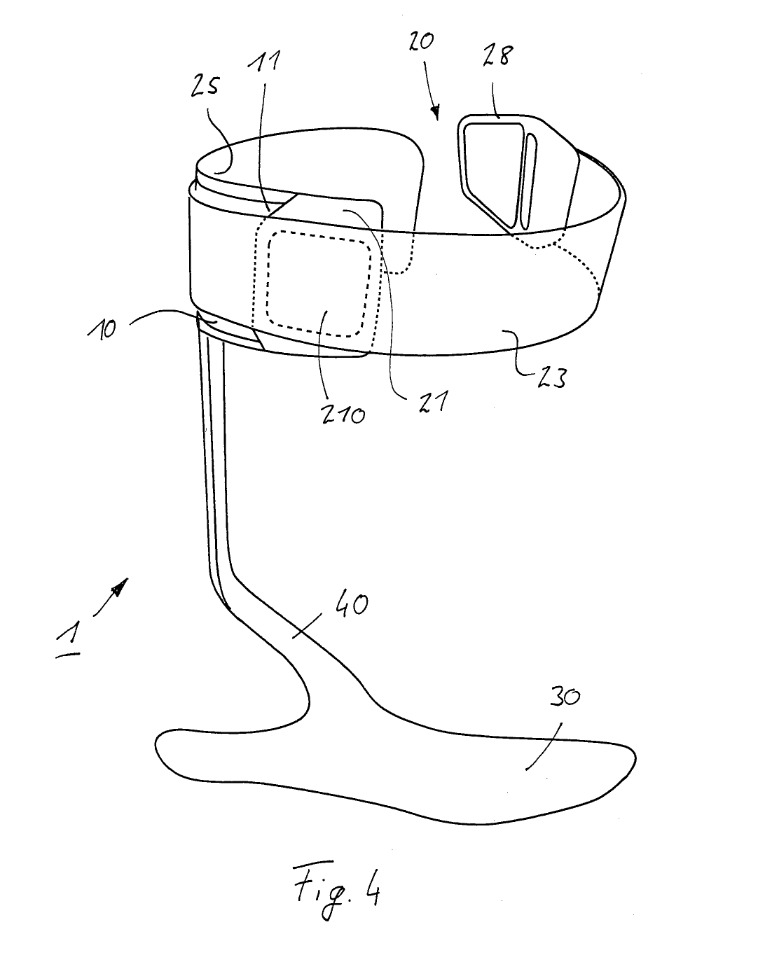

FIG. 3 shows a variant of the invention;

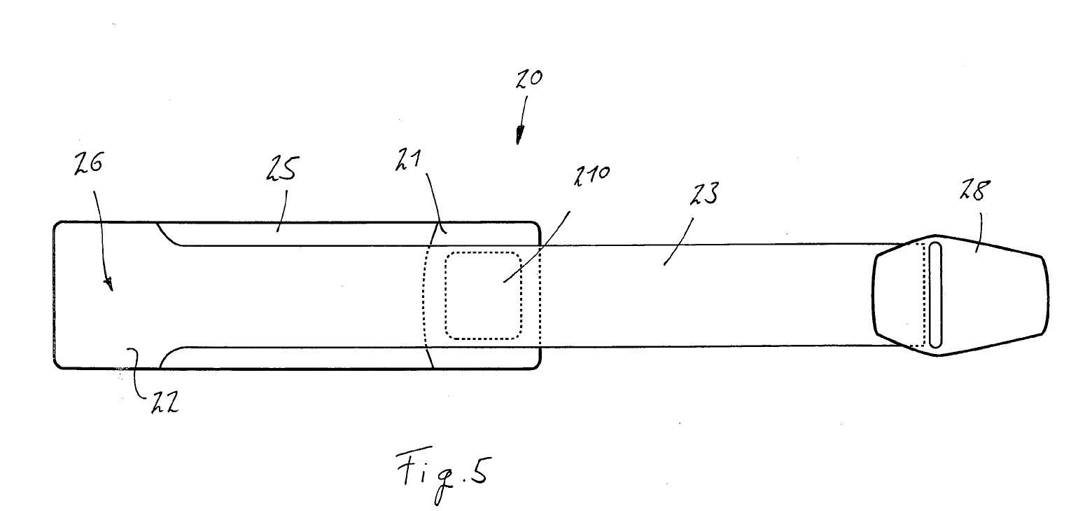

FIG. 4 shows an orthosis with dorsal supporting region; and

FIG. 5 shows a fastening means on its own.

An orthosis in the form of an ankle/foot orthosis (AFO) is shown in FIG. 1, with a supporting region 10, a fastening means 20, and a set-down region 30, which is connected to the supporting region 10 via a connection element 40. In the illustrative embodiment shown, the connection element 40 is designed as a dimensionally stable and substantially rigid element, which permits only a slight movement of the supporting region 10 relative to the set-down region 30. As an alternative to this, provision is mainly made that the set-down region 30 and the supporting region 10 are coupled to each other via an articulated connection, which is designed, for example, as a spring in which the main deformation in the sagittal plane, upon loading in the anterior-posterior direction, takes place in a region around the natural ankle joint. The set-down region 30 is designed as a flat sole plate, which substantially follows the outer contour of a foot or of a shoe. The illustrative embodiment shown is in one piece and made from a fiber-reinforced plastic.

In the fitted state, the supporting region 10 bears on the shin of the orthosis user and at least partially encloses the shin medially and laterally. The contour of the supporting region 10 is substantially gutter-shaped or cup-shaped and can have recesses or apertures to permit improved ventilation of the shin area. The fastening means 20 is arranged on the inner side of the supporting region 10 facing toward the orthosis user in the fitted state, said fastening means 20 at the same time being provided with a padding 25, such that the hard, cup-like structure of the supporting region 10 does not lie directly on the lower leg.

Receiving regions 11, 12 in the form of projections or tongues (not visible in FIG. 1) are arranged both medially and also laterally on the supporting region 10. The receiving regions 11, 12 or projections are formed at the proximal end and distal end of the supporting region 10 and, when the orthosis is in the fitted state on the orthosis user, face toward the rear. The receiving regions 11, 12 are referred to below as projections, which term is also meant to include a configuration in which structures adjoining the supporting region 10 are suitable for being received in pockets 21, 22 in order to bring about a form-fit securing of the padding. In FIG. 2, the orthosis 1 according to FIG. 1 is shown in an open, non-fitted state, in which the two receiving regions 12 formed as projections from a vertical line of the supporting region 10 are also shown by a broken line on the medial side. Corresponding receiving regions 11 and pockets 21 are provided on the lateral side of the supporting region 10.

On the fastening means 20, pockets 21, 22 are formed into which the projections 11, 12 are pushed. By pushing the projections 11, 12 into the pockets 21, 22, the fastening means 20 is secured on the supporting region 10. Provision is made here that the lateral pockets 21 are deeper than the medial pockets 22, and provision is likewise made that the lateral projections 11 are longer than the medial projections 12, wherein the depth of the pockets 21, 22 corresponds to the lengths of the projections 11, 12. The illustrative embodiment provides two pockets 21, 22 and two projections 11, 12 on the medial and lateral sides, respectively, which are offset relative to each other in the longitudinal extent, such that two proximal and two distal pairs of pockets 21, 22 and projections 11, 12 result. Alternatively, it is also possible for just one pocket to be provided medially and laterally and to be secured on a corresponding receiving region.

Connection elements 23, 24 are provided on the fastening means 20, both at the proximal edge and also at the distal edge of the supporting region 10, via which connection elements 23, 24 the orthosis 1 is secured on the limb. The connection elements 23, 24 are secured, for example welded, adhesively bonded or sewn, on the medial side on the fastening means 20 to form the medial pockets 22. From the medial pocket 22, the connection element 23, 24 designed as a strap is guided externally along the outer side of the supporting region 10 and is secured externally on the lateral pocket 21, for example by a velcro fastener. In the case of an elastic configuration of the strap-like connection element 23, 24, the medial and lateral pockets 21, 22 are pretensioned toward each other in the circumferential direction, such that the forwardly open pockets 21, 22 cannot slide down from the rearwardly pointing projections 11, 12. To further secure the orthosis 1, the respective connection element 23, 24 is then guided rearward around the limb (not shown) from the lateral side to the medial side and is secured there on a fixing means 26, 27. The connection element 23, 24 is thus guided once around the orthosis and the limb, wherein the free end is secured on the outer side of the medial pocket. At the free end of the connection element 23, 24, locking elements 28, 29 are secured, for example in the form of detachable velcro elements, which can be secured on the outer side of the locking element 23, 24 or on fleeced areas of the fixing means 26, 27 externally on the medial pockets 22. The securing on the connection elements 23, 24 and on the fixing means 26, 27 is reversible. The connection elements 23, 24 can be flexible and nonelastic, flexible and elastic, or elastic only in parts, and it is likewise possible that the connection element 23, 24 is secured reversibly on the fastening means 20, for example by velcro fasteners.

In the fitted state as shown in FIG. 1, the connection element 23, 24 bridges the gap in the cup-like supporting region 10 from lateral to medial. The supporting region 10 can permit a deformation of the projections 11, 12 toward each other, such that an adjustment to the dimensions of the limb on which the orthosis is to be fitted can take place. The adjustment can be made via the circumferential force applied by the connection elements 23, 24.

The embodiment shown is especially advantageous for patients who are paralyzed on one side or weakened on one side. When the connection element 23 is guided from the medial side to the lateral side, stretched and secured with tensioning on a lateral pocket 21, the hand that is unaffected by the weakness can be used to safely secure the orthosis by then guiding the connection element 23, 24 behind the limb and locking it on the medial side.

The fastening means 20 can also be designed as a one-part element or as a multi-part, permanently interconnected element that has a cushioning function. The medial and lateral projections 11, 12 form self-adaptive side wings which adapt automatically to the shape of the limb under the circumferential force that is applied by the connection element 23, 24.

With Y-shaped velcro tapes 28, 29 as hook areas, it is possible to easily shorten the connection elements 23, 24 designed as straps, by removing the Y-shaped velcro tapes from the fleeced connection elements 23, 24, shortening them to the desired length and then reapplying them.

Particularly in the case of stroke patients, it must be noted that they are able to fit the orthosis using only the unaffected side of the body. By virtue of the connection elements 23, 24 being arranged laterally and being guided from lateral to medial in order to secure the orthosis, it is possible for the orthosis user, in a seated position, to grasp the connection element 23, 24 laterally, using his or her unaffected side, and to simply pull it in the medial direction and secure it there on the hook and loop areas 26, 27 provided there.

FIG. 3 shows a variant of the invention. The supporting region 10 and the set-down region 30 are connected to each other by a separate connection element 40. In addition to a flat sole, the set-down region 30 provides edge elevations 35, which prevent medial and lateral slipping of the foot from the set-down region 30. In the illustrative embodiment shown, the elevations 35 are formed starting in the metatarsal region and extend into the heel region in order, if appropriate, to avoid slipping toward the rear. The connection element 40 is designed as a spring in order to permit a movement of the supporting region 10 relative to the set-down region 30, for example when walking, wherein the spring action of the connection element 40 has the effect that the foot of the orthosis user is moved back to the starting position after unloading. The fastening means 20 according to Figures and 2 is not shown in the illustrative embodiment according to FIG. 3. In contrast to the medial and lateral receiving regions 11, 12, which are designed as projections or tongues, the whole of the rearwardly facing edge of the supporting region 10 in the illustrative embodiment according to FIG. 3 is designed as a receiving region 11, 12. The medial and lateral pocket 22, 21 of the fastening means 20 (not shown) therefore receives an edge of the supporting region 10 extending the full height, such that two pockets 21, 22 in all are present on the lateral side of the fastening means 20 and receive the rearwardly facing ends of the supporting region 10. The connection elements 23, 24 can be arranged at different heights of the fastening means, that is to say a proximal connection means 23 and a distal connection means 24. It is also possible in principle that only one connection means is present which, from the medial side, is guided across the front of the supporting region and the lateral side of the supporting region and around the back of the lower leg in order then to be secured once again on the lateral side of the fastening means 20.

FIG. 4 shows a further variant of the invention in which the orthosis 1 has a set-down region 30 in the form of a flat sole for a foot or shoe, a spring element as connection element 40, and a supporting region 10 arranged dorsally, such that the supporting region 10 bears on the calf and not on the shin of the orthosis user. The orthosis 1 is formed in one piece in the illustrative embodiment shown, but it is possible in principle that the connection element 40, the set-down region 30 and the supporting region 10 are formed separately and are connected to one another to assemble the orthosis 1.

The padding 25 is arranged on the inner side of the supporting region 10 facing toward the user, which padding 25 lies in front of the U-shaped, forwardly open supporting region 10 and cushions the possibly sharp edges of the dimensionally stable material. The padding 25 can protrude past the edges of the supporting region 10.

The connection element 40 is arranged medially on the set-down region and runs obliquely in the dorsal and proximal direction, i.e. obliquely rearward and upward, from the plantar arch and is guided upward along the back of the leg, with the ankle region advantageously being left free.

As in the embodiments described above, the fastening means 20 is secured via pockets 21, 22 on receiving regions 11, 12 of the supporting region 10. Only the lateral receiving region 11 and the lateral pocket 21 are shown in FIG. 4. On the medial side, the front end of the supporting region 10 is inserted in a pocket of the fastening means 20. The medial pocket is composed of the padding 25 and of the connection element 23 sewn, welded or adhesively bonded onto the latter. The connection element 23 is then guided dorsally around the outer side of the supporting region 10 and fixed on a velcro fastener 210 on the outer side of the lateral pocket 21. This fixing can take place with pretensioning of the elastic connection element 23 such that the fastening means 20 is secured safely and permanently on the supporting regions 11 via the pockets 21. The then still open configuration for insertion of the lower leg is then closed by the connection element 23, which is guided across the shin of the orthosis user and is secured with the locking element 28 on the medial outer side of the fastening means 20, for example on a fleeced area on the outer side of the medial pocket.

FIG. 5 shows the fastening means 20 on its own and laid out flat. The padding 25 as base is provided with the connection element 23 on the outer side. The connection element 23 has, at its left-hand end in FIG. 5, a widened region corresponding to the width of the padding 25. In the area of this widened region, the connection element 23 is secured on the padding 25 at the edges, such that a pocket 22 for receiving the receiving region (not shown) is formed. A fixing means 26 in the form of a velcro fastener or of a fleeced area can be arranged and configured on the outer side of the pocket 22, so as to secure on the outer side thereof the locking element 28 fastened at the opposite end.

From the pocket 22, the connection element 23 extends in the direction of the other end of the padding 25, where a separate pocket 21 is formed by the sewing-on, welding-on or adhesive bonding of a textile or the like. On the outer side of the pocket 21, which is arranged laterally in the fitted state, a closure element 210 in the form of a velcro fastener is applied, secured or formed, in order to secure thereon the connection element 23 provided, for example, with a fleeced layer. The locking element 23, which is designed as a Y-shaped element, is secured on the end of the connection element 23 via a further velcro fastener, such that the connection element 23 provided on both sides with a fleeced layer can be received and secured by the closure elements designed as hook elements on the locking element 28.

1. An orthosis, comprising:
a supporting region includes a fastening member to fit the orthosis on a limb receiving regions oriented opposite to each other, and pockets to receive the receiving regions, the pockets being arranged on the fastening member.

2. The orthosis as claimed in claim 1, wherein the fastening member is part of a padding which is arranged on a side of the orthosis facing toward the user.

3. The orthosis as claimed in claim 1, wherein the receiving regions and pockets are arranged medially and laterally.

4. The orthosis as claimed in claim 3, wherein the laterally arranged pockets and receiving regions are longer than the medially arranged pockets and receiving regions.

5. The orthosis as claimed in claim 1, wherein the supporting region has a cup-like or gutter-like shape.

6. The orthosis as claimed in claim 1, wherein the pockets are connected to each other via a reversibly attachable connection element.

7. The orthosis as claimed in claim 6, wherein the connection element is elastic and allows the pockets to be pretensioned toward each other.

8. The orthosis as claimed in claim 6, wherein the connection element is part of the fastening member.

9. The orthosis as claimed in claim 6, wherein a fixing arrangement for the connection element is arranged on the outer side of at least one of the pockets.

10. The orthosis as claimed in claim 6, wherein the connection element is designed as a strap looping around the supporting region and the limb.

11. The orthosis as claimed in claim 1, wherein hook and loop regions are present on the fastening member.

12. The orthosis as claimed in claim 1, wherein the connection element forms a lateral to medial bridge across a gap between the receiving regions.

13. The orthosis as claimed in claim 1, wherein the supporting region has a longitudinal extent and is designed elastically about an axis parallel to the longitudinal extent.

14. The orthosis as claimed in claim 1, wherein a set-down region of the orthosis is connected to the supporting region and bears on the limb.

15. The orthosis as claimed in claim 14, wherein the set-down region and the supporting region are connected to each other in an articulated manner.

16. The orthosis as claimed in claim 1, wherein the receiving regions are designed as projections.

17. An orthosis, comprising:
a supporting region configured to contact a limb;
a fastening member to secure the orthosis to the limb;
receiving regions oriented opposite to each other on the supporting region;
pockets to receive the receiving regions, the pockets being arranged on the fastening member.

18. The orthosis as claimed in claim 17, wherein the fastening member is part of a padding, the padding being arranged on a side of the orthosis facing toward the user.

19. The orthosis as claimed in claim 17, wherein the receiving regions and pockets are arranged medially and laterally on the supporting region.

20. The orthosis as claimed in claim 19, wherein the laterally arranged pockets and receiving regions are longer than the medially arranged pockets and receiving regions.
